(19) United States
(12) Patent Application Publication
(10) Pub. No.: US 2023/0087361 A1
(43) Pub. Date: Mar. 23, 2023

(54) USE OF DIANTHRONE COMPOUND IN PREPARATION OF MEDICAMENT FOR PREVENTION AND/OR TREATMENT OF MYOCARDIAL ISCHEMIC DISEASE AND RELATED DISEASE THEREOF

(71) Applicants: National Institutes for Food and Drug Control, Beijing (CN); Institute of Materia Medica Chinese Academy of Medical Sciences, Beijing (CN)

(72) Inventors: Shuangcheng MA, Beijing (CN); Hua SUN, Beijing (CN); Feng WEI, Beijing (CN); Jianbo YANG, Beijing (CN); Ting OUYANG, Beijing (CN); Qi WANG, Beijing (CN); Zihan CHEN, Beijing (CN); Ying WANG, Beijing (CN); Yunfei SONG, Beijing (CN); Zhiwei CHEN, Beijing (CN); Huiyu GAO, Beijing (CN); Xueting WANG, Beijing (CN)

(73) Assignees: National Institutes for Food and Drug Control, Beijing (CN); Institute of Materia Medica Chinese Academy of Medical Sciences, Beijing (CN)

(21) Appl. No.: 17/931,608

(22) Filed: Sep. 13, 2022

(30) Foreign Application Priority Data
Sep. 14, 2021 (CN) 202111072549.8

(51) Int. Cl. A61K 31/122; A61P 9/10

(52) U.S. Cl. CPC A61K 31/122; A61P 9/10

(57) ABSTRACT

A dianthrone compound may be used in preparation of a medicament for prevention and/or treatment of myocardial ischemic diseases and related diseases thereof. The dianthrone compound includes trans-emodin dianthrone and/or cis-emodin dianthrone.

### CROSS REFERENCE TO RELATED APPLICATION

This patent application claims the benefit and priority of Chinese Patent Application No. 202111072549.8, filed with the China National Intellectual Property Administration on Sep. 14, 2021, the disclosure of which is incorporated by reference herein in its entirety as part of the present application.

### TECHNICAL FIELD

The present disclosure relates to the technical field of biological medicine, in particular to use of a dianthrone compound in preparation of a medicament for prevention and/or treatment of myocardial ischemic diseases and related diseases thereof.

### BACKGROUND

Cardiovascular disease is a major disease threatening human life and health and has become the single largest cause of death in the world. In 2014, the death toll from cardiovascular disease accounted for about 30% of the total global deaths. With the advancement of society and the improvement of people's living standards, the prevalence rate of cardiovascular diseases in low- and middle-income countries is increasing at an alarming rate, and this change is accelerating. In China, the incidence and mortality of cardiovascular diseases show a continuous upward trend in recent years, and the age of onset gradually becomes younger. Among cardiovascular diseases, myocardial ischemic heart disease is the focus of cardiovascular diseases, which can develop into angina pectoris, arrhythmia, myocardial infarction (MI), and heart failure. It is the main cause of disability and death worldwide and seriously harms human health. Feasible and effective alleviation of the injury caused by myocardial ischemia has become a hot issue facing the medical industry today.

Myocardial ischemia is a pathological state in which coronary arterial blood flow is reduced, myocardial oxygen supply is reduced, myocardial energy metabolism is abnormal, and normal working of the heart cannot be supported for various reasons. Common causes include coronary atherosclerosis (95-99%), as well as inflammation (rheumatic inflammation, syphilitic inflammation, Kawasaki disease and thronboangitis obliterans), spasm, embolism, spontaneous coronary artery dissection, connective tissue disease, trauma, congenital malformations, and the like. The pathogenesis is believed to be related to cell apoptosis caused by myocardial ischemia, calcium overload, oxidative stress, and inflammation.

Dianthrone compounds have many pharmacological effects such as antidepressant, anti-tumor and antiviral effects, but there is no report that dianthrone compounds are used to prepare drugs for the prevention and/or treatment of myocardial ischemic diseases.

### SUMMARY

An objective of the present disclosure is to provide use of a dianthrone compound in preparation of a medicament for prevention and/or treatment of myocardial ischemic diseases and related diseases thereof

To achieve the above objective, the present disclosure provides the following technical solution:

The present disclosure provides use of a dianthrone compound in preparation of a medicament for prevention and/or treatment of myocardial ischemic diseases and related diseases thereof;

the dianthrone compound includes trans-emodin dianthrone and/or cis-emodin dianthrone.

Preferably, if the dianthrone compound includes the trans-emodin dianthrone and the cis-emodin dianthrone, the trans-emodin dianthrone and the cis-emodin dianthrone may have a molar ratio of 1:(0.5-1).

Preferably, the trans-emodin dianthrone and the cis-emodin dianthrone may have a molar ratio of 56:38.

Preferably, the myocardial ischemic diseases and related diseases thereof may include one or more of coronary heart disease (CHD), arrhythmia, heart failure, MI, and angina pectoris.

Preferably, a mass percentage of the dianthrone compound in the medicament may be 0.1%-95%.

Preferably, the mass percentage of the dianthrone compound in the medicament may be 30%-90%.

Preferably, the medicament may be in the pharmaceutical dosage form of an oral preparation, a topical preparation, an injection, or a cavity administration preparation.

Preferably, the oral preparation may include a tablet, a capsule, a pill, a granule, an oral liquid, or a suspension.

Preferably, the injection may include a water injection, a powder injection or an infusion.

The present disclosure provides use of a dianthrone compound in preparation of a medicament for prevention and/or treatment of myocardial ischemic diseases and related diseases thereof. The dianthrone compound includes trans-emodin dianthrone and/or cis-emodin dianthrone. The present disclosure provides the use of a dianthrone compound in preparation of a medicament for prevention and/or treatment of myocardial ischemic diseases and related diseases thereof. Results of the example show that compared with diltiazem, the dianthrone compound has the following remarkable activity in pharmacology: significantly improving the ST-segment elevation of mice with myocardial ischemia on electrocardiogram (ECG), reducing the level of a serum biomarker for myocardial injury, cardiac troponin T (cTn-T), lowering the level of myocardial enzyme, and improving the state of myocardial ischemic injury.

### BRIEF DESCRIPTION OF THE DRAWINGS

FIG. 1 shows effects of ST segment changes of mice with isoproterenol (ISO) induced myocardial ischemia on lead II ECG (n=4), (*P<0.05 versus the blank control group; ^{#}/³<0.05 versus the model group;);

FIG. 2 shows effects of serum biomarker for myocardial injury cTn-T in mice with ISO-induced myocardial ischemia in (n=7-10) (*P<0.05 versus the blank control group; 4/3<0.05 versus the model group;);

FIG. 3 shows effects of serum myocardial enzyme CK-MB in mice with ISO-induced myocardial ischemia (n=7-10), (*P<0.05 versus the blank control group; 4/3<0.05 versus the model group;).

### DETAILED DESCRIPTION OF THE EMBODIMENTS

The present disclosure provides use of a dianthrone compound in preparation of a medicament for prevention and/or treatment of myocardial ischemic diseases and related diseases thereof;

the dianthrone compound includes trans-emodin dianthrone and/or cis-emodin dianthrone.

In the present disclosure, the trans-emodin dianthrone (HSW-W-25) is represented by a chemical structural formula as shown in a formula II; the cis-emodin dianthrone (HSW-W-26) is represented by a chemical structural formula as shown in a formula II;

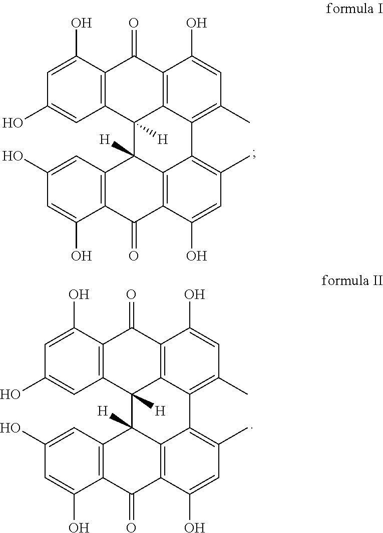

There is no particular limitation on the sources of the cis-emodin dianthrone and the trans-emodin dianthrone in the present disclosure, which can either be commercially available or prepared according to methods known in the art.

In the present disclosure, the trans-emodin dianthrone may be a racemate mixture of the trans-emodin dianthrone; the cis-emodin dianthrone may be a mesomer mixture of the cis-emodin dianthrone.

In the present disclosure, if the dianthrone compound includes the trans-emodin dianthrone and the cis-emodin dianthrone, the trans-emodin dianthrone and the cis-emodin dianthrone may preferably have a molar ratio of 1:(0.5-1), and more preferably 56:38.

In the present disclosure, the myocardial ischemic diseases and related diseases thereof may preferably include one or more of CHD, arrhythmia, heart failure, MI, and angina pectoris.

In the present disclosure, a mass percentage of the dianthrone compound in the medicament may preferably be 0.1%-95%, more preferably 30%-90%, and most preferably 50%-80%.

In the present disclosure, the medicament may preferably be in the pharmaceutical dosage form of an oral preparation, a topical preparation, an injection, or a cavity administration preparation.

In the present disclosure, the oral preparation may preferably include a tablet, a capsule, a pill, a granule, an oral liquid, or a suspension.

In the present disclosure, the injection may preferably include a water injection, a powder injection or an infusion.

In the present disclosure, the dosage form of the drug preferably includes a gas dosage form, a liquid dosage form, a solid dosage form, or a semi-solid dosage form.

In the present disclosure, the gas dosage form may preferably include an aerosol or a spray.

In the present disclosure, the liquid dosage form may preferably include a solution, an emulsion, a suspension, an injection, an eye drop, a nasal drop, a lotion, or a liniment. In the present disclosure, the solution may preferably include a true solution or a colloidal solution; the emulsion may preferably include an 01W type emulsion or a W10 type emulsion; the injection may preferably include a water injection, a powder injection, or an infusion.

In the present disclosure, the solid dosage form may preferably include a tablet, a capsule, a granule, a powder, a pellet, a dropping pill, a suppository, a film, a patch, or a powder inhalation; in the present disclosure, the tablet may preferably include a conventional tablet, an enteric-coated tablet, a buccal tablet, a dispersible tablet, a chewable tablet, an effervescent tablet, or an oral disintegrating tablet. In the present disclosure, the capsule may preferably include a hard capsule, a soft capsule, or an enteric-coated capsule.

In the present disclosure, the semi-solid dosage form may preferably include an ointment, a gel, or a paste.

In the present disclosure, the dosage form of the medicament may preferably include an ordinary preparation, a sustained-release preparation, a controlled-release preparation, a targeting preparation, or a particulate drug delivery system.

In the present disclosure, the medicament may further preferably include pharmaceutically acceptable excipients.

In the present disclosure, if the dosage form of the medicament is preferably a tablet, the excipient may preferably include one or more of a diluent, a binder, a wetting agent, a disintegrant, a lubricant, and a glidant.

In the present disclosure, the diluent may preferably include one or more of starch, dextrin, sucrose, glucose, lactose, mannitol, sorbitol, xylitol, microcrystalline cellulose, calcium sulfate, calcium hydrogen phosphate, and calcium carbonate; the wetting agent may preferably include one or more of water, ethanol, and isopropanol; the binder may preferably include one or more of starch paste, dextrin, syrup, honey, glucose solution, microcrystalline cellulose, acacia mucilage, gelatin mucilage, sodium carboxymethyl cellulose, methylcellulose, hydroxypropyl methyl cellulose, ethyl cellulose, acrylic resin, carbomer, polyvinylpyrrolidone, and polyethylene glycol (PEG); the disintegrant may preferably include one or more of dry starch, microcrystalline cellulose, low-substituted hydroxypropyl cellulose, crosslinked polyvinylpyrrolidone, crosslinked sodium carboxymethyl cellulose, sodium carboxymethyl starch, sodium bicarbonate, citric acid, polyoxyethylene sorbitol fatty acid esters, and sodium dodecyl sulfate; the lubricant may preferably include one or more of talc powder, silica, stearates, tartaric acid, liquid paraffin, and PEG; the glidant may preferably include one or more of talc powder, silica, stearates, tartaric acid, liquid paraffin, and PEG.

In the present disclosure, the tablet may preferably include a coated tablet, and the coated tablet may preferably include a sugar-coated tablet, a film-coated tablet, or an enteric-coated tablet.

In the present disclosure, the tablet may preferably include a single-layer tablet, or a multilayer tablet.

In the present disclosure, if the dosage form of the medicament is preferably a capsule, the excipient may preferably include one or more of a diluent, a glidant, a wetting agent, a binder, and a disintegrant.

In the present disclosure, f the dosage form of the medicament is preferably an injection, a solvent of the injection may preferably include one or more of water, ethanol, isopropanol, and propylene glycol; the excipient of the injection may preferably include one or more of a solubilizer, a solubilizing assistant, a pH regulator, and an osmotic pressure regulator. The solubilizer may preferably include one or more of poloxamer, lecithin, and hydroxypropyl-P-cyclodextrin, and the solubilizing assistant may preferably include one or more of poloxamer, lecithin, and hydroxypropyl-P-cyclodextrin; the pH regulator may preferably include one or more of phosphate, acetate, hydrochloric acid, and sodium hydroxide; the osmotic pressure regulator may preferably include one or more of sodium chloride, mannitol, glucose, phosphate, and acetate.

In the present disclosure, if the dosage form of the medicament is preferably a freeze-dried powder injection, the excipient may preferably include a proppant. The proppant may preferably include mannitol and/or glucose.

In the present disclosure, the excipient of the medicament may further preferably include one or more of a coloring agent, a preservative, a perfume, and a flavoring agent.

There is no particular limitation on administration methods of the medicament in the present disclosure, as long as the administration methods are well-known in the art.

In the present disclosure, a route of administration of the medicament may preferably include enteral administration or parenteral administration. The enteral administration may preferably include oral administration or rectal administration; the parenteral administration may preferably include intravenous injection, intramuscular injection, subcutaneous injection, intranasal, oral transmucosal, transocular, intrapulmonary and transrespiratory tract, transdermal, or intravaginal administration.

In the present disclosure, a daily dose of the medicament may preferably be 0.001-150 mg/kg body weight, more preferably 0.1-100 mg/kg body weight, further preferably 1-60 mg/kg body weight, and most preferably 2-30 mg/kg body weight; the above-mentioned dose may be administered in one dose unit or divided into several dose units, depending on doctor's clinical experience and a dosing regimen including use of other treatments.

The medicament provided by the present disclosure may be administered alone or in combination with other therapeutic or symptomatic drugs. If the medicament provided by the present disclosure has a synergistic effect with other therapeutic drugs, the dose thereof should be adjusted as the case may be.

The technical solution of the present disclosure will be described below clearly and completely in conjunction with the examples of the present disclosure. Apparently, the described examples are only a part of, not all of, the examples of the present disclosure. Based on the examples of the present disclosure, all other examples obtained by those of ordinary skill in the art without creative efforts shall fall within the protection scope of the present disclosure.

The following examples and pharmacological activity experiments are intended to further illustrate the present disclosure, but these should not mean any limitation to the present disclosure. The experimental methods without specific conditions in the following examples are selected in accordance with conventional methods and conditions, or in accordance with the product specification.

In the following examples, the full names or corresponding Chinese names of some substances are as follows:

HSW-W-25: Trans-emodin dianthrone

HSW-W-26: Cis-emodin dianthrone

HSW-W-25/26: Trans-emodin dianthrone and cis-emodin dianthrone (abbreviated as dianthrone)

Control: Blank control

Model: Model control

ISO: Isoprenaline

CK-MB: Creatine kinase-isoenzyme

cTn-T: Cardiac troponin T

The room temperature in the following examples is as conventionally described in the art, and generally refers to 15-25° C.

Experimental example 1 HSW-W-25/26 (Dianthrone) activity assay for the mouse model of ISO-induced myocardial ischemia

Experimental method:

Male Kunming mice were acclimatized and randomly divided into four groups: a blank control group, an ISO model group, an ISO+diltiazem 20 mg.kg¹ positive control group, and an ISO+dianthrone 10 mgkgi group. Mice in the dianthrone group were intragastrically given the corresponding dose of dianthrone once a day, those in the diltiazem group were intraperitoneally given once a day, and those in the blank control group and the model group were given the same dose of solvent (both of which were 10 mL. kg) for 10 consecutive days. On day 4 of administration, the mice in each group were injected subcutaneously with 40 mg.kg¹ ISO once; the concentration of ISO subcutaneous injection was adjusted to 20 mg.kg⁻ on days 5 to 10 of administration. All mice were modeled at 2 h after administration. On the final day, the animals were treated at 2 h after ISO administration and modeling. Animal weights were recorded during the experiment. The final weight of the blank control group was 33.2±1.9 g, that of the model group was 30.7±1.9 g, and that of the HSW-W-25/26 (dianthrone) group was 29.6±2.1 g. The animals were in good condition and there was no death.

ECG monitoring: The day before the end of the experiment, four mice were randomly selected from each group, and the lead II ECGs were recorded for the analysis of the ST-segment elevation 0.5 h after modeling.

Assay for biomarker for myocardial injury and myocardial enzyme: The blood was drawn from the orbit of the mouse, let stand at room temperature for 2 h, and centrifuged at 3,500 rpm for 20 min to separate the serum. The serum levels of CK-MB and cTn-T were detected according to the ELISA kit instructions.

Experimental results:

1. The effect of HSW-W-25/26 (dianthrone) on the lead II ECG of the mice with ISO-induced myocardial ischemia

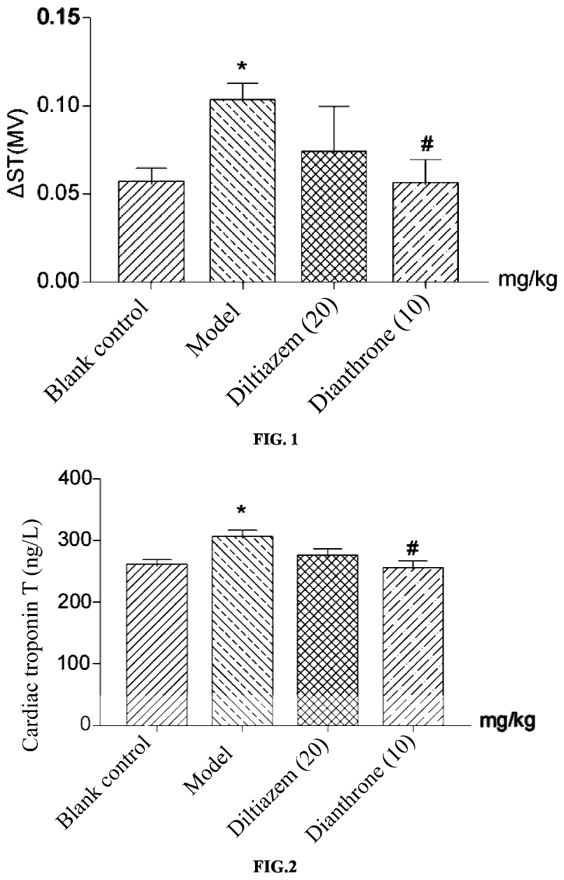

Electrocardiography is the most commonly used non-invasive method for diagnosing myocardial ischemia. The results are shown in FIG. 1. After ISO administration, a continuous convex ST-segment elevation was induced on the mouse ECG, which showed typical myocardial ischemic changes. The degree of ST-segment elevation (AST) was calculated, and the ST-segment was elevated from 0.06±0.02 MV to 0.10±0.02 MV. The results are shown in Table 1. Compared with the blank control group, the ISO model group showed a significant increase. The ISO-induced ST-segment elevation on ECG could be significantly reduced by 10 mg/kg HSW-W-25/26 (dianthrone), which was statistically different from the model group. The activity was better than that of the positive control drug, 20 mg/kg diltiazem.

2. The effect of HSW-W-25/26 (dianthrone) on the serum biomarker for myocardial injury cTn-T in the mice with ISO-induced myocardial ischemia

When myocardial necrosis occurs, some protein substances contained in the myocardium are released from the myocardial tissue and appear in the peripheral blood circulation, which can be used as a judgment index of myocardial injury. Troponin (Tn) is a protein that regulates myocardial tissue contraction. There are differences in molecular structure and immunology between cardiac troponin (cTn) and Tn in the skeletal muscle. Therefore, cTn is unique to the myocardium and is the most specific and sensitive marker of choice for judging myocardial necrosis. The cTn has three subtypes: cTnT, cTnI, and cTnC. cTn-T has very high diagnostic value for both early and late myocardial necrosis.

The results are shown in FIG. 2 and Table 1. ISO caused a significant increase in serum biomarker for myocardial injury, cTn-T, in mice, which was statistically different from that of the blank control group. 10 mg/kg HSW-W-25/26 (dianthrone) could significantly reduce the serum cTn-T level, and the serum cTn-T was reduced from 298.10±37.21 ng/L to 253.04±32.73 ng/L, which was more active than 20 mg/kg diltiazem.

3. The effect of HSW-W-25/26 (dianthrone) on the serum myocardial enzyme CK-MB level in the mice with ISO-induced myocardial ischemia

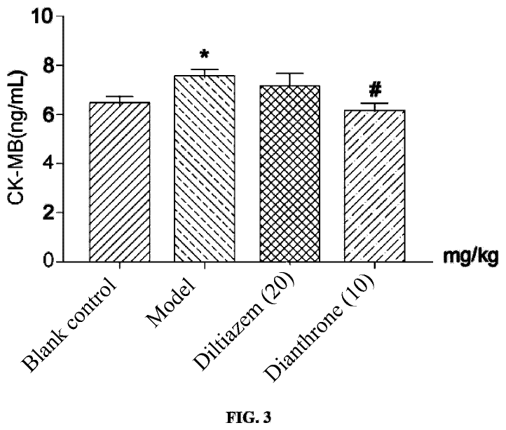

Serum creatine kinase-isoenzyme (CK-MB) is another clinically specific and sensitive index for judging myocardial necrosis. The results are shown in FIG. 3 and Table 1. ISO could cause a significant increase in serum CK-MB activity in mice, which was statistically different from that of the blank control group. 10 mg/kg HSW-W-25/26 (dianthrone) significantly reduced the serum CK-MB level raised by ISO, and the CK-MB level was reduced from 7.56±1.05 ng/mL to 6.11±0.81 ng/mL, which was more active than 20 mg/kg diltiazem.

TABLE 1
HSW-W-25/26 activity assay for the mouse
model of ISO-induced myocardial ischemia

| No. | ΔST/MV | cTn-T/ng/L | CK-MB/ng/mL |
|---|---|---|---|
| Blank control | 0.06 ± 0.02 | 258.88 ± 30.43 | 6.40 ± 0.93 |
| Model | 0.10 ± 0.02 | 298.10 ± 37.21 | 7.56 ± 1.05 |
| Diltiazem | 0.07 ± 0.05 | 273.93 ± 36.17 | 7.11 ± 1.63 |
| Dianthrone | 0.06 ± 0.03 | 253.04 ± 32.73 | 6.11 ± 0.81 |

The results from Table 1 and FIGS. 1 to 3 show that compared with diltiazem, the dianthrone compound has the following remarkable activity in pharmacology: significantly improving the ST-segment elevation of mice with myocardial ischemia on ECG, reducing the level of the serum biomarker for myocardial injury cTn-T, lowering the level of myocardial enzyme, and improving the state of myocardial ischemic injury.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A method for preventing and/or treating patients with myocardial ischemic diseases and related diseases, the method comprising:
providing patients a medicament comprising a dianthrone compound; wherein the dianthrone compound comprises trans-emodin dianthrone and/or cis-emodin dianthrone.

2. The method according to claim 1, wherein the dianthrone compound comprises the trans-emodin dianthrone and the cis-emodin dianthrone, and the trans-emodin dianthrone and the cis-emodin dianthrone have a molar ratio of 1:(0.5-1).

3. The method according to claim 2, wherein the trans-emodin dianthrone and the cis-emodin dianthrone have a molar ratio of 56:38.

4. The method according to claim 1, wherein the myocardial ischemic diseases and related diseases thereof comprise one or more of coronary heart disease (CHD), arrhythmia, heart failure, myocardial infarction (MI), and angina pectoris.

5. The method according to claim 2, wherein the myocardial ischemic diseases and related diseases thereof comprise one or more of coronary heart disease (CHD), arrhythmia, heart failure, myocardial infarction (MI), and angina pectoris.

6. The method according to claim 3, wherein the myocardial ischemic diseases and related diseases thereof comprise one or more of coronary heart disease (CHD), arrhythmia, heart failure, myocardial infarction (MI), and angina pectoris.

7. The method according to claim 1, wherein a mass percentage of the dianthrone compound in the medicament comprising the dianthrone compound is 0.1%-95%.

8. The method according to claim 7, wherein the mass percentage of the dianthrone compound in the medicament comprising the dianthrone compound is 30%-90%.

9. The method according to claim 1, wherein the medicament comprising the dianthrone compound is in the pharmaceutical dosage form of an oral preparation, a topical preparation, an injection, or a cavity administration preparation.

10. The method according to claim 9, wherein the oral preparation comprises a tablet, a capsule, a pill, a granule, an oral liquid, or a suspension.

11. The method according to claim 9, wherein the injection comprises a water injection, a powder injection or an infusion.
